(19) United States
(12) Patent Application Publication
(10) Pub. No.: US 2025/0099255 A1
(43) Pub. Date: Mar. 27, 2025

(54) ANKLE ARTHROPLASTY DEVICES, SYSTEMS, AND METHODS

(71) Applicant: Vilex in Tennessee, Inc., McMinnville, TN (US)

(72) Inventors: Beat HINTERMANN, Liestal (CH); Daniel J. TRIPLETT, Smithfield, UT (US); Brock JOHNSON, Draper, UT (US); Roxa Angelina Ruiz Wong, Liestal (CH); Zachary C. CHRISTENSEN, Wellsville, UT (US); Nathan A. STEVENS, Marana, AZ (US)

(21) Appl. No.: 18/372,410

(22) Filed: Sep. 25, 2023

(51) Int. Cl. A61F 2/42; A61B 17/16; A61F 2/30

(52) U.S. Cl. CPC A61F 2/4202; A61B 17/1682; A61F 2002/30401; A61F 2002/3093; A61F 2002/30985; A61F 2002/4207

(57) ABSTRACT

The adjustable system for ankle arthroplasty may comprise a baseplate with a baseplate bone facing side configured to be secured to a talus and a baseplate joint facing side. The system may further have an articulating component with an articulating component joint facing side with a prosthetic talar articular surface and an articulating component bone facing side configured to adjustably engage the baseplate joint facing surface. The baseplate and the articulating component may cooperate to define a locking mechanism configured to be actuated to secure the articulating component bone facing side to the baseplate joint facing side.

### TECHNICAL FIELD

The present disclosure relates to devices, systems, and methods for joint prostheses. More specifically, the present disclosure relates to devices, systems, and methods for providing orthopedic implants for the restoration of anatomical mobility of joints, for example, an ankle joint.

### BACKGROUND

The present disclosure relates to systems, devices, and methods for ankle arthroplasty. Ankle arthroplasty has gained significant attention as an effective solution for patients suffering from debilitating ankle joint disorders such as osteoarthritis, rheumatoid arthritis, and traumatic injuries. Traditional ankle arthroplasty implants have contributed to improved joint function and alleviation of pain in numerous cases. However, these conventional implants often lack the versatility required to cater to the diverse anatomical variations and functional demands of individual patients.

The success of ankle arthroplasty hinges on achieving optimal joint kinematics and stability, both of which are essential for restoring natural gait patterns and facilitating a pain-free range of motion. Conventional fixed implants offer limited options for adjustment, leaving little room for customization to suit the unique anatomical and biomechanical characteristics of each patient. This lack of adjustability can result in suboptimal implant placement, joint instability, reduced implant longevity, and ultimately hindered patient outcomes.

Accordingly, there is a need for improved ankle arthroplasty devices, systems, and methods. Such devices, systems, and methods would address the current limitations of fixed implants by allowing orthopedic surgeons to fine-tune implant positioning, alignment, and joint dynamics. This level of adjustability would enable the surgeon to adapt the device to the specific joint characteristics and functional needs of each patient, thereby optimizing joint stability, enhancing joint kinematics, and potentially extending device longevity.

### SUMMARY

The various systems and methods of the present disclosure have been developed in response to the present state of the art, and in particular, in response to the problems and needs in the art that have not yet been fully solved by currently available ankle arthroplasty systems and methods.

In some embodiments, an adjustable system for ankle arthroplasty may comprise a baseplate comprising, a baseplate bone facing side configured to be secured to a talus and a baseplate joint facing side. The system may further comprise an articulating component comprising, an articulating component joint facing side comprising a prosthetic talar articular surface and an articulating component bone facing side configured to adjustably engage the baseplate joint facing surface. The baseplate and the articulating component cooperate to define a locking mechanism configured to be actuated to secure the articulating component bone facing side to the baseplate joint facing side.

In the adjustable system of any preceding paragraph, the baseplate bone facing side may be additively manufactured with integrated porous features configured for bone integration.

In the adjustable system of any preceding paragraph, the articulating component may be configured for both right ankle arthroplasty and left ankle arthroplasty.

In the adjustable system of any preceding paragraph, the locking mechanism may include a polymer anti-rotation component.

In some embodiments, an adjustable system for ankle arthroplasty may comprise a baseplate comprising, a baseplate bone facing side configured to be secured to a talus and a baseplate joint facing side. The adjustable system for ankle arthroplasty may further comprise an articulating component comprising, an articulating component joint facing side comprising a prosthetic talar articular surface and an articulating component bone facing side configured to be secured to the baseplate joint facing side at any of a plurality of relative positions.

In the adjustable system of any preceding paragraph, the articulating component bone facing side may be configured to be secured to the baseplate joint facing side at any of a plurality of relative positions in-vivo and in-vitro.

In the adjustable system of any preceding paragraph, the articulating component bone facing side may be configured to be secured to the baseplate joint facing side at any of a plurality of relative positions along an anterior-posterior direction.

In the adjustable system of any preceding paragraph, the baseplate joint facing side may comprise a plurality of first angled surfaces and the articulating component bone facing side may comprise a plurality of second angled surfaces configured to slidably engage the plurality of first angled surfaces such that the articulating component is centered on the baseplate in a medial-lateral direction.

In some embodiments, an adjustable system for ankle arthroplasty may comprise a baseplate comprising a baseplate bone facing side configured to be secured to one of a talus and a tibia and a baseplate joint facing side. The adjustable system for ankle arthroplasty may further comprise an articulating component configured to be secured to the baseplate such that the articulating component can be moved along the baseplate without detaching the articulating component from the baseplate. The articulating component may comprise an articulating component joint facing side comprising a prosthetic articular surface and an articulating component bone facing side.

In the adjustable system of any preceding paragraph, the baseplate bone facing side further may comprise one or more protrusions configured to engage a bone.

In the adjustable system of any preceding paragraph, the baseplate and the articulating component may cooperate to define a locking mechanism configured to secure the articulating component to the baseplate. The locking mechanism may comprise a plurality of baseplate locking features of the baseplate and a plurality of articulating component locking features of the articulating component. Wherein the plurality of articulating component locking features may be configured to slidably mate with the plurality of baseplate locking features at any of a plurality of relative positions of the baseplate and the articulating component along an anterior-posterior direction.

In the adjustable system of any preceding paragraph, the plurality of baseplate locking features may comprise a first baseplate dovetail feature that may be adjustably positionable along the anterior-posterior direction and a second baseplate dovetail feature that may be adjustably positionable, independently of a position of the first baseplate dovetail feature, along the anterior-posterior direction.

In the adjustable system of any preceding paragraph, the plurality of baseplate locking features further may comprise a first actuator configured to slidably adjust the first baseplate dovetail feature and a second actuator configured to slidably adjust the second baseplate dovetail feature.

In the adjustable system of any preceding paragraph, the first actuator and the second actuator may be configured to be captive within the baseplate.

In the adjustable system of any preceding paragraph, the first actuator and the second actuator may comprise threaded features.

In the adjustable system of any preceding paragraph, the plurality of baseplate locking features further may comprise an actuator locking mechanism configured to lock a position of the first actuator and the second actuator.

In the adjustable system of any preceding paragraph, the actuator locking mechanism may be configured to captively snap into the baseplate.

In the adjustable system of any preceding paragraph, the baseplate may comprise one or more apertures configured to facilitate visualization of the plurality of baseplate locking features when the plurality of baseplate locking features are in a locked position.

In the adjustable system of any preceding paragraph, the articulating component may comprise one or more apertures configured to facilitate visualization through the articulating component in a medial-lateral direction.

In the adjustable system of any preceding paragraph, an adjustable system for ankle arthroplasty may further comprise a size trial comprising a plurality of apertures configured to receive a cutting instrument.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1 illustrates a perspective view of an adjustable system for ankle arthroplasty according to an embodiment of the present disclosure;

FIG. 2 illustrates a perspective view of the adjustable system for ankle arthroplasty of FIG. 1 implanted in an ankle joint;

FIG. 3 illustrates an exploded perspective view of the adjustable system for ankle arthroplasty of FIG. 1;

FIG. 4 illustrates a perspective view of an articulating component of the adjustable system for ankle arthroplasty of FIG. 1;

FIG. 5 illustrates a side sectional view of the adjustable system for ankle arthroplasty of FIG. 1;

FIG. 6A illustrates a side view of an adjustable system for ankle arthroplasty according to an embodiment of the present disclosure;

FIG. 6B illustrates a side view of an adjustable system for ankle arthroplasty according to an embodiment of the present disclosure;

FIG. 7 illustrates a side view of an adjustable system for ankle arthroplasty according to an embodiment of the present disclosure;

FIG. 8A illustrates a side view of a pair of actuators according to an embodiment of the present disclosure;

FIG. 8B illustrates an exploded perspective view of an actuator and a locking nut according to an embodiment of the present disclosure;

FIG. 9A illustrates a perspective view of a locking feature according to an embodiment of the present disclosure;

FIG. 9B illustrates a perspective view of a baseplate according to an embodiment of the present disclosure;

FIG. 10 illustrates a top view of a baseplate according to an embodiment of the present disclosure;

FIG. 11A illustrates a front sectional view of an adjustable system for ankle arthroplasty according to an embodiment of the present disclosure;

FIG. 11B illustrates a front sectional view of an articulating component according to an embodiment of the present disclosure;

FIG. 11C illustrates a bottom view of an articulating component according to an embodiment of the present disclosure;

FIG. 12 illustrates a front sectional view of a baseplate according to an embodiment of the present disclosure;

FIG. 13A illustrates an exploded perspective view of an adjustable system for ankle arthroplasty according to an embodiment of the present disclosure;

FIG. 13B illustrates a front view of an actuator according to an embodiment of the present disclosure;

FIG. 13C illustrates a partial sectional side view of the adjustable system for ankle arthroplasty of FIG. 13A;

FIG. 13D illustrates a partial front view of the adjustable system for ankle arthroplasty of FIG. 13A;

FIGS. 14A thru 14D illustrate bottom perspective views of an adjustable system for ankle arthroplasty according to various embodiments of the present disclosure;

FIG. 15A illustrates a side view of an adjustable system for ankle arthroplasty according to an embodiment of the present disclosure;

FIG. 15B illustrates a side sectional view of the adjustable system for ankle arthroplasty of FIG. 15A;

FIG. 16 illustrates perspective views of a baseplate according to various embodiments of the present disclosure;

FIG. 17 illustrates bottom perspective views of a baseplate according to various embodiments of the present disclosure;

FIG. 18 illustrates a front view of an adjustable system for ankle arthroplasty according to an embodiment of the present disclosure;

FIG. 19 illustrates a partial side view of the adjustable system for ankle arthroplasty of FIG. 18;

FIG. 20 illustrates top views of a trialing and peg drilling & keel reaming instrument according to various embodiments of the present disclosure;

FIG. 21 illustrates a side view of a drill; a guide wire; and a trialing and peg drilling & keel reaming instrument according to an embodiment of the present disclosure;

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to ankle arthroplasty devices, systems, and methods. Those skilled in the art will recognize that the following description is merely illustrative of the principles of the technology, which may be applied in various ways to provide many alternative embodiments. The present disclosure illustrates ankle joints for the purposes of illustrating the concepts of the present design. However, it will be understood that other variations and uses are contemplated including, but not limited to, applications in the arm, wrist, finger, toe, spine, pelvis, any other bone or joint, etc.

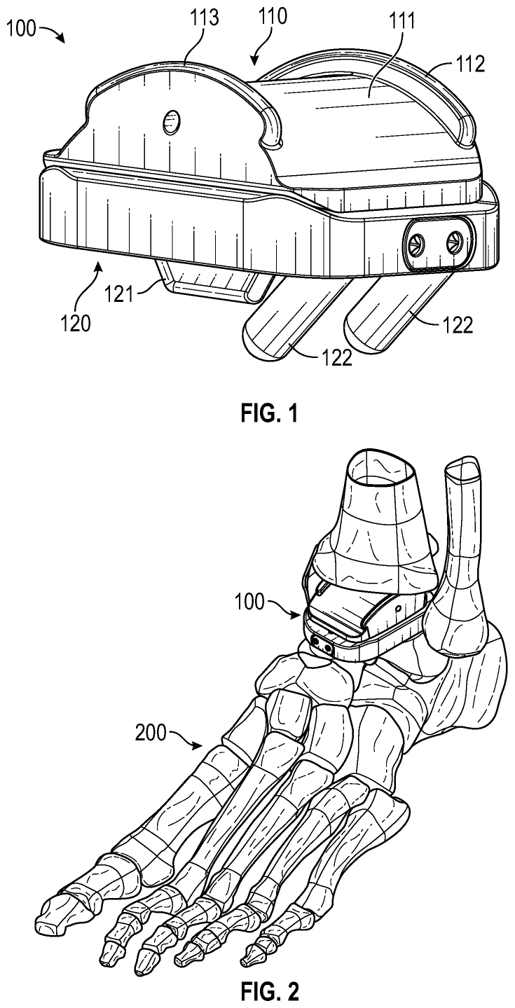

FIG. 1 illustrates a perspective view of an adjustable system for ankle arthroplasty 100 according to an embodiment. In some embodiments the adjustable system for ankle arthroplasty 100 may include a baseplate 120 and an articulating component 110. The baseplate 120 may comprise a baseplate bone facing side configured to be secured to a talus and a baseplate joint facing side. The articulating component 110 may comprise an articulating component joint facing side comprising a prosthetic talar articular surface 111 and an articulating component bone facing side configured to adjustably engage the baseplate joint facing surface. The baseplate 120 and the articulating component 110 may cooperate to define a locking mechanism configured to be actuated to secure the articulating component bone facing side to the baseplate joint facing side. The baseplate 120 may further comprise a joint facing side configured to adjustably engage the articulating component 110.

In some embodiments the adjustable system for ankle arthroplasty 100 may include an articulating component 110. The articulating component 110 may comprise a joint facing side comprising a prosthetic talar articular surface 111 and a bone facing side configured to adjustably engage a baseplate 120 joint facing side. The joint facing side of the articulating component 110 may further include a first articular surface edge 112 and a second articular surface edge 113. The first articular surface edge 112 and the second articular surface edge 113 may be configured to engage an inferior articular surface of a tibia such that the inferior articular surface is centered on the articulating component 110 in a medial-lateral plane. The inferior articular surface of the tibia may be comprised of a bone portion or an artificial implant.

FIG. 2 illustrates a perspective view of an adjustable system for ankle arthroplasty 100 implanted in an ankle joint of a skeletal foot 200. In some embodiments the adjustable system for ankle arthroplasty 100 may be configured for both right ankle arthroplasty and left ankle arthroplasty.

In some embodiments, an adjustable system for ankle arthroplasty 100 may comprise a baseplate 120 and an articulating component 110. The baseplate 120 may comprise a baseplate bone facing side configured to be secured to a talus and a baseplate joint facing side. The articulating component 110 may comprise an articulating component joint facing side comprising a prosthetic talar articular surface 111 and an articulating component bone facing side configured to be secured to the baseplate joint facing side at any of a plurality of relative positions. In an embodiment, the articulating component bone facing side may be configured to be secured to the baseplate joint facing side at any of a plurality of relative positions in-vivo and in-vitro.

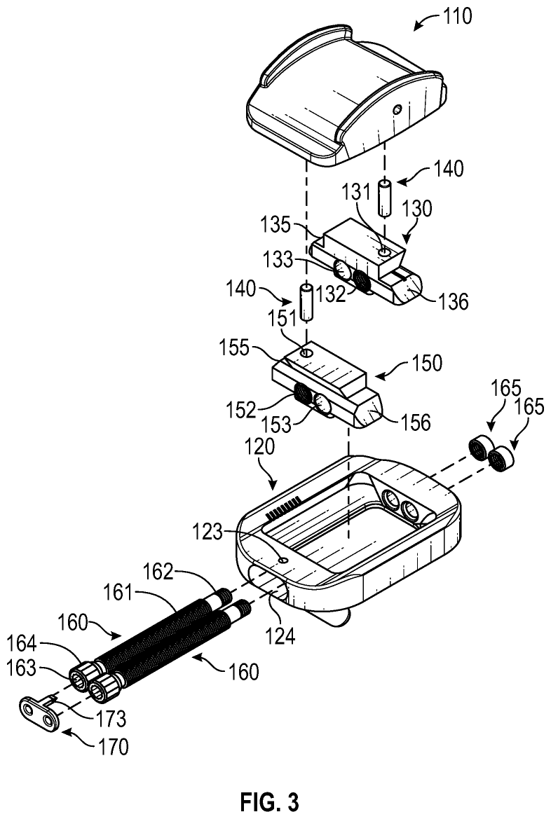

FIG. 3 illustrates an exploded perspective view of an adjustable system for ankle arthroplasty 100. In some embodiments the adjustable system for ankle arthroplasty 100 may include a baseplate 120; an articulating component 110; and one or more actuators 160 configured to engage a plurality of locking features. The one or more actuators 160 may be configured to engage one or more locking nuts 165. The one or more locking nuts 165 may be configured to captively engage the one or more actuators 160 within the baseplate 120 while enabling the one or more actuators 160 to rotate.

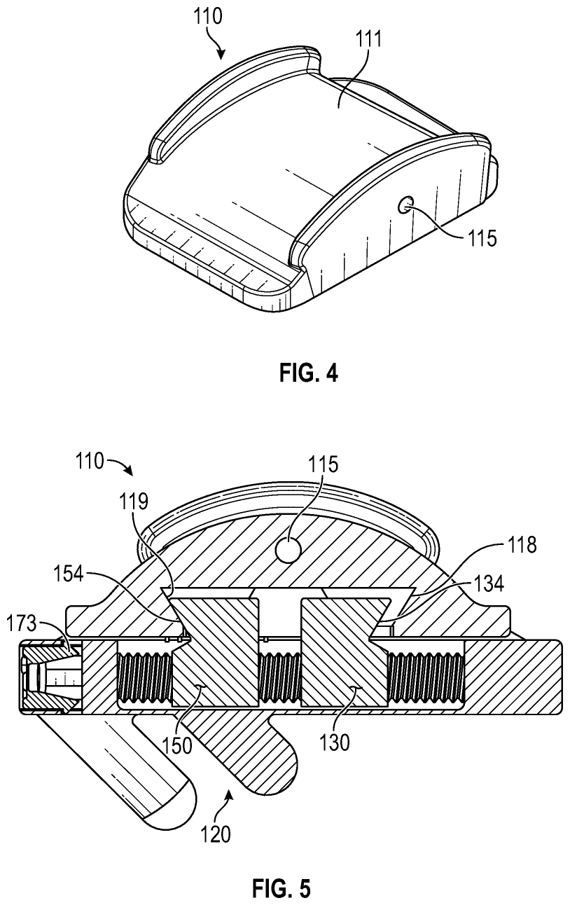

FIG. 4 illustrates a perspective view of an articulating component 110 of an adjustable system for ankle arthroplasty 100 according to an embodiment. The articulating component 110 may comprise a prosthetic talar articular surface 111 configured to engage an inferior articular surface of a tibia. The prosthetic talar articular surface 111 may comprise substantially curved surface configured to slidably engage an inferior articular surface of a tibia and facilitate rotation of the inferior articular surface in an anterior-posterior plane.

FIG. 5 illustrates a side sectional view of an adjustable system for ankle arthroplasty 100 according to an embodiment. The adjustable system for ankle arthroplasty 100 may comprise a baseplate 120 and an articulating component 110, wherein the baseplate 120 and the articulating component 110 cooperate to define a locking mechanism configured to secure the articulating component 110 to the baseplate 120. The locking mechanism may comprise a plurality of baseplate locking features of the baseplate 120 and a plurality of articulating component locking features of the articulating component 110. The plurality of articulating component locking features may be configured to slidably mate with the plurality of baseplate locking features at any of a plurality of relative positions of the baseplate 120 and the articulating component 110 along an anterior-posterior direction.

In some embodiments, the plurality of baseplate locking features comprises a first locking feature 130 that may be adjustably positionable along an anterior-posterior direction and a second locking feature 150 that may be adjustably positionable, independently of a position of the first locking feature 130, along an anterior-posterior direction. In some embodiments, the first locking feature 130 may be configured as a baseplate dovetail feature, including a first engagement surface 134. The second locking feature 150 may be configured as a baseplate dovetail feature, including a second engagement surface 154.

In some embodiments, the articulating component 110 may comprise a third engagement surface 118 and a fourth engagement surface 119. The third engagement surface 118 may be configured to engage the first engagement surface 134 in a manner that may result in a locking force being applied to the articulating component 110 in both a posterior direction and a caudal direction. The fourth engagement surface 119 may be configured to engage the second engagement surface 154 in a manner that may result in a locking force being applied to the articulating component 110 in both an anterior direction and a caudal direction.

In some embodiments, an adjustable system for ankle arthroplasty 100 may comprise a baseplate 120 and an articulating component 110. The baseplate 120 may comprise a baseplate bone facing side configured to be secured to one of a talus and a tibia and a baseplate joint facing side. The articulating component 110 may comprise an articulating component joint facing side comprising a prosthetic articular surface and an articulating component bone facing side. The articulating component 110 may be configured to be secured to the baseplate 120 such that the articulating component 110 can be moved along the baseplate 120 without detaching the articulating component 110 from the baseplate 120. In an embodiment, the articulating component bone facing side may be configured to be secured to the baseplate joint facing side at any of a plurality of relative positions along an anterior-posterior direction.

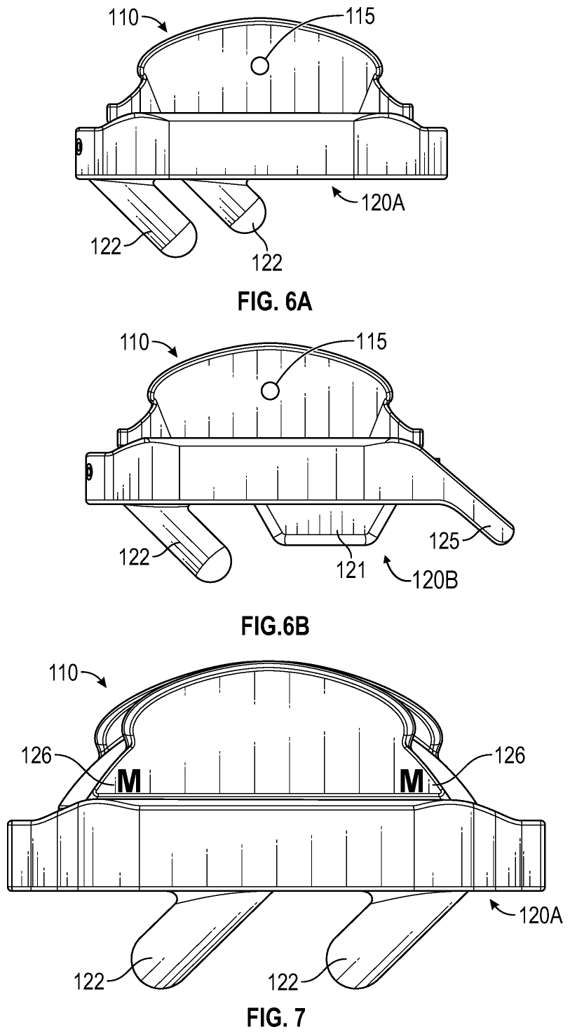

FIG. 6A and FIG. 6B show side views of an adjustable system for ankle arthroplasty 100 according to various embodiments. The adjustable system for ankle arthroplasty 100 may comprise a baseplate 120 including a baseplate bone facing side comprising one or more protrusions of various configurations configured to engage a bone portion. The various configurations may be configured to complement a range of patient anatomies. The adjustable system for ankle arthroplasty 100 may comprise a baseplate 120A and an articulating component 110. The baseplate 120A may comprise one of more talar pegs 122. In another embodiment, the adjustable system for ankle arthroplasty 100 may comprise a baseplate 120B and an articulating component 110. The baseplate 120B may comprise one or more talar pegs 122, a keel 121 and a tail 125. The tail 125 may be configured to engage an anterior portion of a talus. In some embodiments, the baseplate 120 may include at least one of talar peg 122, keel 121 and tail 125. In other embodiments, the baseplate 120 may not include any protrusions on the baseplate bone facing side.

FIG. 7 illustrates a side view of an adjustable system for ankle arthroplasty 100 according to an embodiment. The adjustable system for ankle arthroplasty 100 may be configured for both right ankle arthroplasty and left ankle arthroplasty. The adjustable system for ankle arthroplasty 100 may comprise a baseplate 120 and an articulating component 110. The baseplate may be configured to be symmetrical about a medial-lateral plane and not symmetrical about an anterior-posterior plane nor a cephalad-caudal plane. The articulating component 110 may be configured to be symmetrical about an anterior-posterior plane and not symmetrical about a medial-lateral plane nor a cephalad-caudal plane. To ensure proper orientation of the articulating component 110 on the baseplate 120, the articulating component 110 may include an orientation indicator 126 to identify the medial side of the articulating component 110.

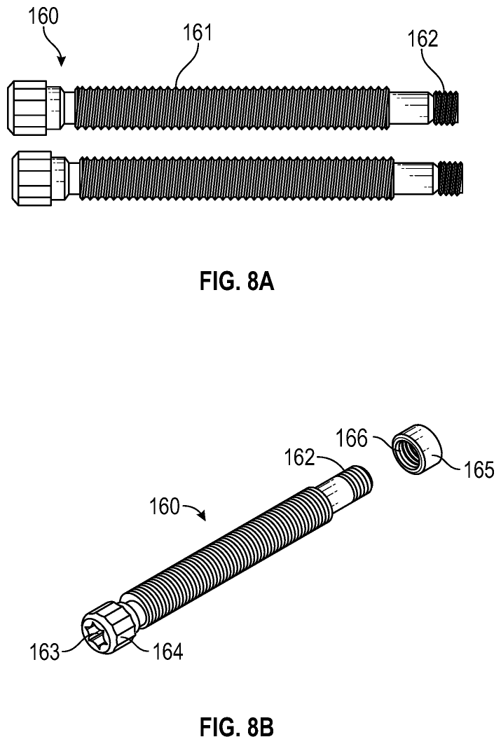

FIG. 8A illustrates a side view of a pair of actuators 160 and FIG. 8B illustrates an exploded perspective view of an actuator 160 and a locking nut 165 according to an embodiment. In an embodiment, the actuator 160 may comprise a first threaded portion 161; a second threaded portion 162; a female drive feature 163; and a faceted surface 164. The locking nut may comprise an internal thread 166. The internal thread 166 and the second threaded portion 162 may be configured to threadably secure the locking nut 165 to the actuator 160. The first threaded portion 161 may be configured to threadably engage a locking feature such that rotation of the actuator 160 results in linear translation of the locking feature. The female drive feature 163 may be configured to receive the working end of a driver tool. The female drive feature 163 may be configured as a hexalobe, hexagon or other non-circular drive feature. The faceted surface 164 may be configured as a non-circular surface; preferably 12 equal sides, alternately, between 6 and 20 equal sides.

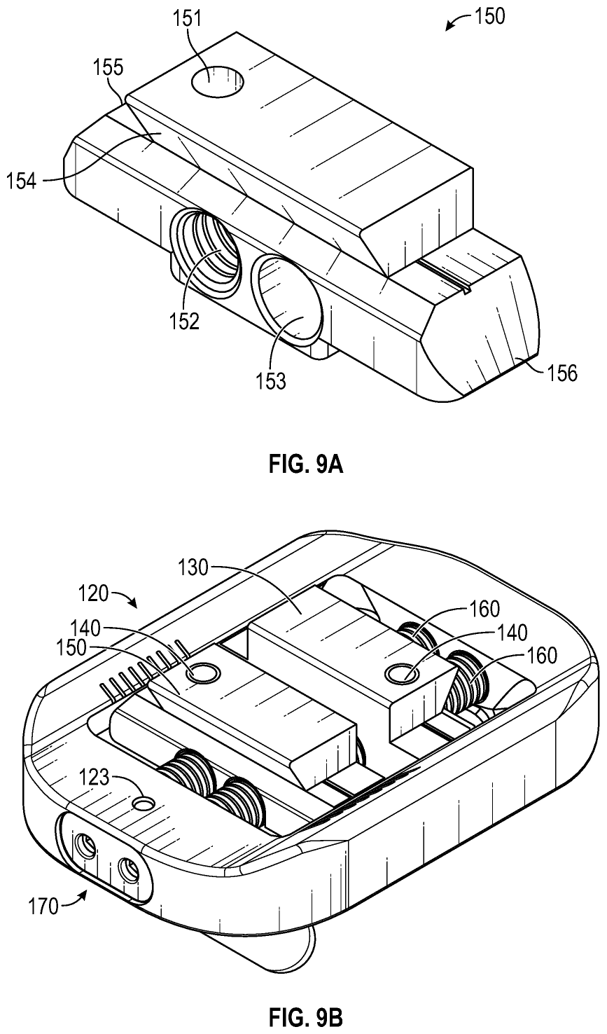

FIG. 9A illustrates a perspective view of a locking feature and FIG. 9B illustrates a perspective view of a baseplate 120 according to an embodiment. A first locking feature 130 may comprise a first anti-rotation hole 131; a first threaded hole 132; a first actuator clearance hole 133; a third retention surface 135; and a fourth retention surface 136. A baseplate 120 may be configured to receive a first locking feature 130; a first actuator 160; a first anti-rotation component 140; a second locking feature 150; a second actuator 160; a second anti-rotation component 140; and an actuator locking mechanism 170. The baseplate 120 may be configured so that the first locking feature 130 is positioned so that the first engagement surface 134 is posterior facing; the first actuator 160 passes through the second actuator clearance hole 153 of the second locking feature 150 without engaging the second actuator clearance hole 153; and the first actuator 160 threadably engages the first threaded hole 132 in the first locking feature 130. The second threaded portion 162 of the first actuator 160 may be threadably engaged to the internal thread 166 of the first locking nut 165 to captively engage the first actuator 160 within the baseplate 120 while still permitting rotation of the first actuator 160. The first anti-rotation component 140 is securably engaged within the first anti-rotation hole 131. The first anti-rotation hole 131 may be configured so that when the first anti-rotation component 140 is engaged, the material of the first anti-rotation component 140 deformably engages the first threaded portion 161 of the first actuator 160 to hinder rotation of the first actuator 160. The first actuator 160 may be configured to slidably adjust the first locking feature 130 through rotation of the first actuator 160.

A second locking feature 150 may comprise a second anti-rotation hole 151; a second threaded hole 152; a second actuator clearance hole 153; a first retention surface 155; and a second retention surface 156. The baseplate 120 may be configured so that the second locking feature 150 is positioned so that the second engagement surface 154 is anterior facing; the second actuator 160 threadably engages the second threaded hole 152 in the second locking feature 150; and the second actuator 160 passes through the first actuator clearance hole 133 of the first locking feature 130 without engaging the first actuator clearance hole 133. The second threaded portion 162 of the second actuator 160 may be threadably engaged to the internal thread 166 of the second locking nut 165 to captively engage the second actuator 160 within the baseplate 120 while still permitting rotation of the second actuator 160. The second anti-rotation component 140 is securably engaged within the second anti-rotation hole 151. The second anti-rotation hole 151 may be configured so that when the second anti-rotation component 140 is engaged, the material of the second anti-rotation component 140 deformably engages the second threaded portion 161 of the second actuator 160 to hinder rotation of the second actuator 160. The second actuator 160 may be configured to slidably adjust the second locking feature 150 through rotation of the second actuator 160. The anti-rotation component 140 may be made using a material that is softer than the material of the actuator 160, preferably PEEK, PEAK, Acetal, Polyamide, other polymer, Titanium, or other biocompatible material.

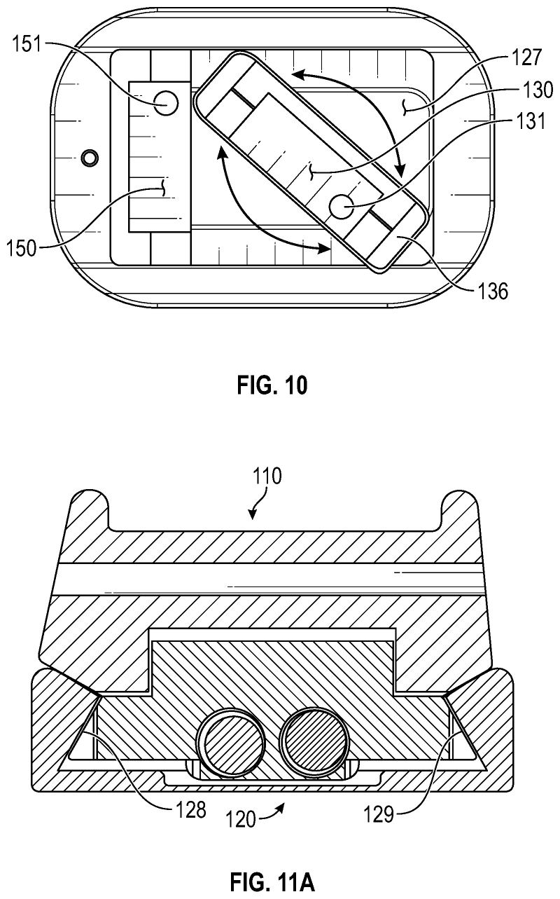

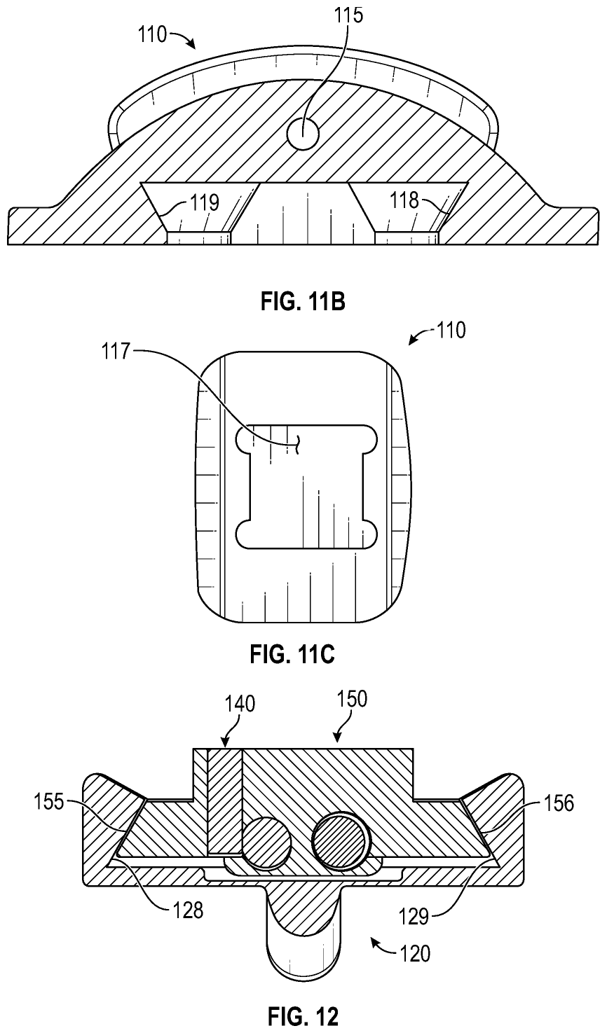

FIG. 10 illustrates a top view of a baseplate 120 and FIG. 11A illustrates a front sectional view of an adjustable system for ankle arthroplasty 100 according to an embodiment. The baseplate 120 may comprise a baseplate cavity 127 configured to receive a first locking feature 130 and a second locking feature 150. The baseplate cavity 127; the first locking feature 130; and the second locking feature 150 may be configured so that when the baseplate 120 is assembled the first locking feature 130 and the second locking feature 150 are slidably translatable in a anterior-posterior direction but captive in a medial-lateral direction and a cephalad-caudal direction. The baseplate cavity 127 may further include a fifth retention surface 128 and a sixth retention surface 129. The baseplate 120 may be configured so that the opening of the baseplate cavity 127 may be smaller than the surfaces created by the fifth retention surface 128 and the sixth retention surface 129. Additionally, a first retention surface 155 of a second locking feature 150 may be configured to slidably engage the fifth retention surface 128. A second retention surface 156 of a second locking feature 150 may be configured to slidably engage the sixth retention surface 129. A third retention surface 135 of a first locking feature 130 may be configured to slidably engage the fifth retention surface 128. A fourth retention surface 136 of a first locking feature 130 may be configured to slidably engage the sixth retention surface 129.

The first locking feature 130 and the second locking feature 150 may be configured so that the first locking feature 130 and the second locking feature 150 may be inserted into the baseplate cavity 127 at an angle and rotated into position such that the first retention surface 155 and the third retention surface 135 engage the fifth retention surface 128; and the second retention surface 156 and the fourth retention surface 136 engage the sixth retention surface 129.

FIG. 11B illustrates a front sectional view of an articulating component 110 and FIG. 11C illustrates a bottom view of an articulating component 110 according to an embodiment. The articulating component 110 may comprise an articulating component cavity 117 on the articulating component bone facing side. The articulating component cavity 117 may comprise a third engagement surface 118 and a fourth engagement surface 119. The third engagement surface 118 and the fourth engagement surface 119 may be configured so that the open of the articulating component cavity 117 is larger than the base of the articulating component cavity 117.

FIG. 12 illustrates a front sectional view of a baseplate 120 according to an embodiment. The baseplate 120 may include a second locking feature 150 comprising a first retention surface 155 and a second retention surface 156. The second locking feature may be configured so that the first retention surface 155 is slidably engaged with a fifth retention surface 128 and the second retention surface 156 is slidably engaged with the sixth retention surface 129. In some embodiments, the retention surfaces may be configured as dovetail features and may result in the locking features being captive within the baseplate 120 in a medial-lateral plane and a cephalad-caudal plane while enabling translation in an anterior-posterior plane.

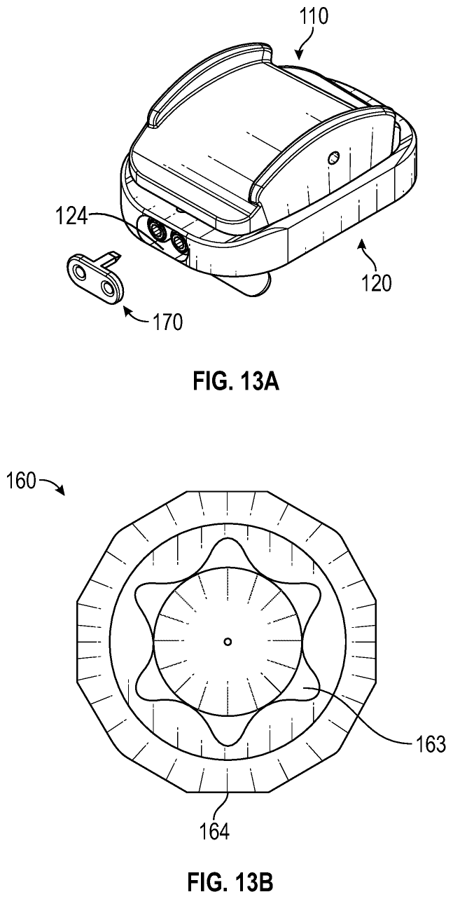

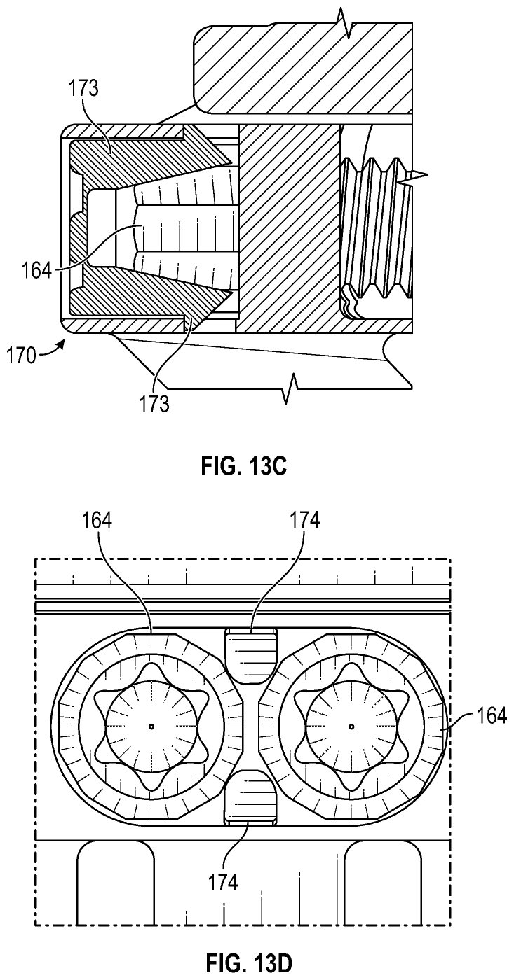

FIG. 13A through FIG. 13D show various features of an actuator locking mechanism 170 of an adjustable system for ankle arthroplasty 100 according to an embodiment. The actuator locking mechanism may be configured to inhibit rotation of the actuator 160. The actuator locking mechanism 170 may be configured to be placed after an articulating component 110 is adjusted to a final location and the locking features have been actuated to secure the position of the articulating component 110 to the baseplate 120. The actuator locking mechanism 170 may comprise an engagement feature 173 and an anti-rotation feature 174. The engagement feature 173 may be configured to captively snap into a baseplate 120 within an actuator pocket 124. The engagement feature 173 may include one or more prongs configured to engage snap holes 123 on the baseplate 120. The anti-rotation feature 174 may be configured to engage one or more faceted surfaces 164 of one or more actuators 160 to prevent rotation of the one or more actuators 160. In an embodiment, the actuator locking mechanism 170 may be configured to lock a position of a first actuator and a second actuator.

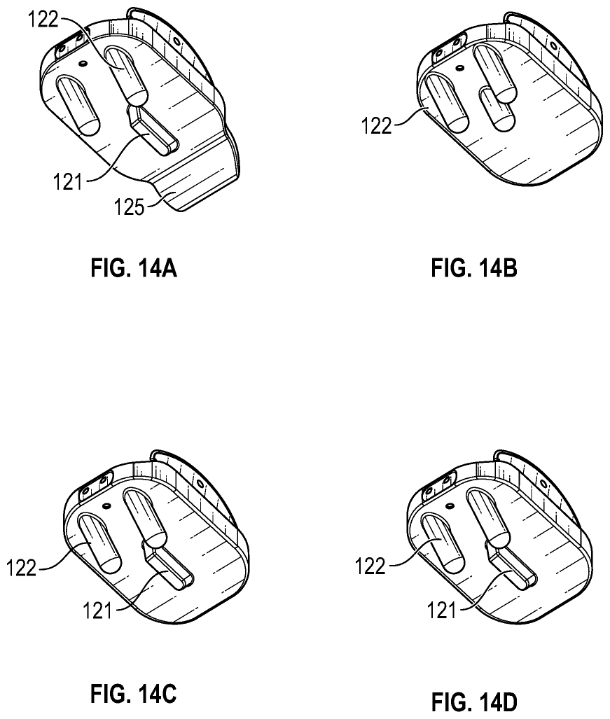

FIGS. 14A thru 14D show bottom perspective views of an adjustable system for ankle arthroplasty 100 according to various embodiments. In some embodiments, the baseplate 120 may comprise a bone facing side comprising a keel 121, a tail 125, and/or one or more talar pegs 122. The keel 121, tail 125, and/or the talar pegs 122 may be configured to engage a bone and may be further configured to secure a baseplate 120 to a talus. The keel 121, tail 125, and/or the talar pegs 122 may further be configured to prevent translation or rotation of a baseplate 120 with respect to a talus. The keel 121 and/or the talar pegs 122 may be configured to be cemented to a talus to provide additional fixation of the baseplate 120 to a talus.

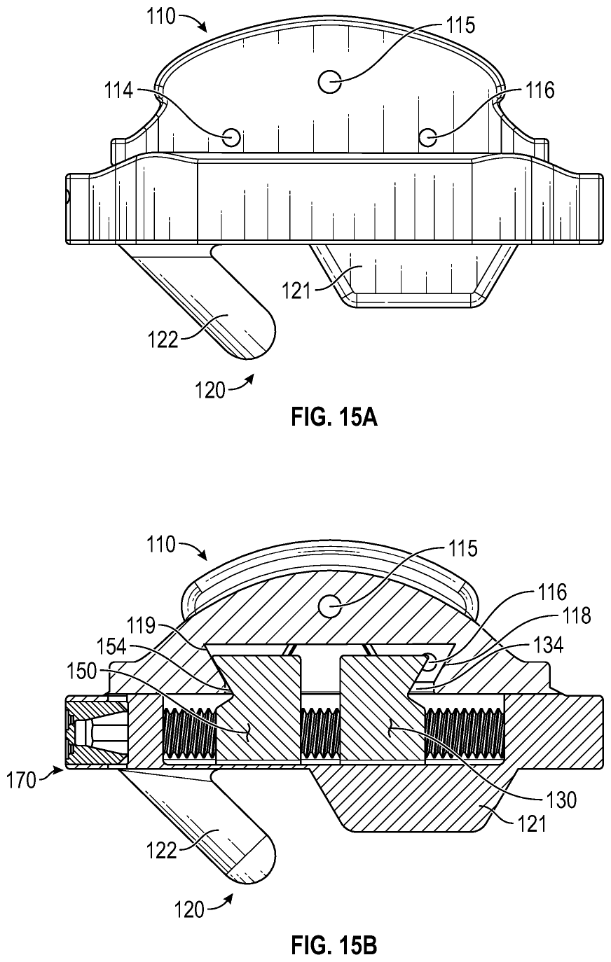

FIG. 15A illustrates a side view and FIG. 15B illustrates a side sectional view of an adjustable system for ankle arthroplasty 100 according to an embodiment. The adjustable system for ankle arthroplasty may be configured to provide a means for confirming the locking features are fully engaged with the articulating component 110. The adjustable system for ankle arthroplasty 100 may comprise an articulating component 110, a first locking feature 130, and a second locking feature 150. The articulating component 110 may include a first visualization aperture 114 and a second visualization aperture 116. The first visualization aperture 114 may be configured so that the aperture is fully obscured when the second locking feature 150 is in a locked position and the second engagement surface 154 is engaged with the fourth engagement surface 119. The second visualization aperture 116 may be configured so that the aperture is fully obscured when the first locking feature 130 is in a locked position and the first engagement surface 134 is engaged with the third engagement surface 118. The articulating component 110 may be configured to facilitate visualization through the articulating component 110 in a medial-lateral direction. Alternately, the first visualization aperture 114 and the second visualization aperture 116 may facilitate visualization through radiographic means. The first visualization aperture 114 and the second visualization aperture 116 may be configured to provide a means of confirming the location of the locking features in an anterior-posterior plane. In an embodiment, the first visualization aperture 114 and the second visualization aperture 116 may be configured to facilitate visualization of the plurality of baseplate locking features when the plurality of baseplate locking features are in a locked position.

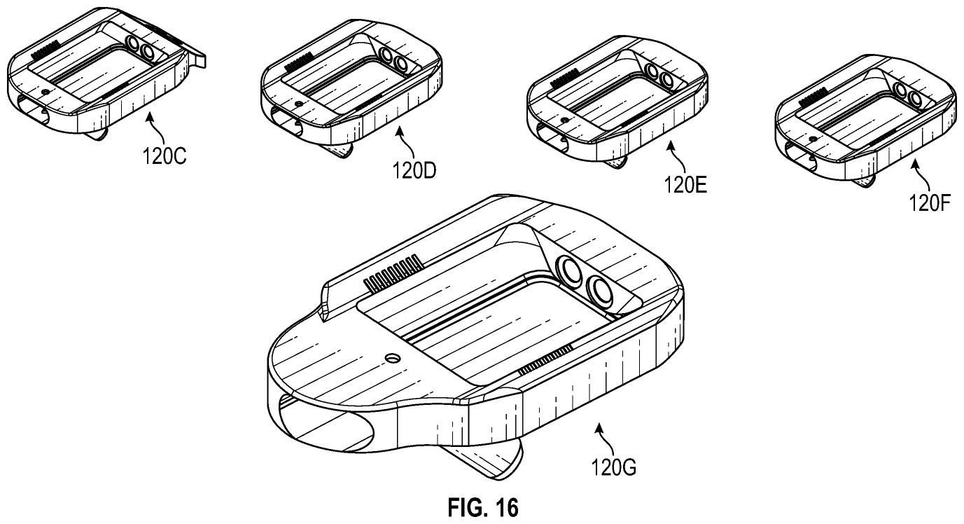

FIG. 16 illustrates perspective views of various baseplates according to various embodiments. The baseplates 120C through 120G may comprise an outer profile and thickness. The outer profile and thickness of the various baseplates may be configured to complement a range of patient anatomies and surgical conditions.

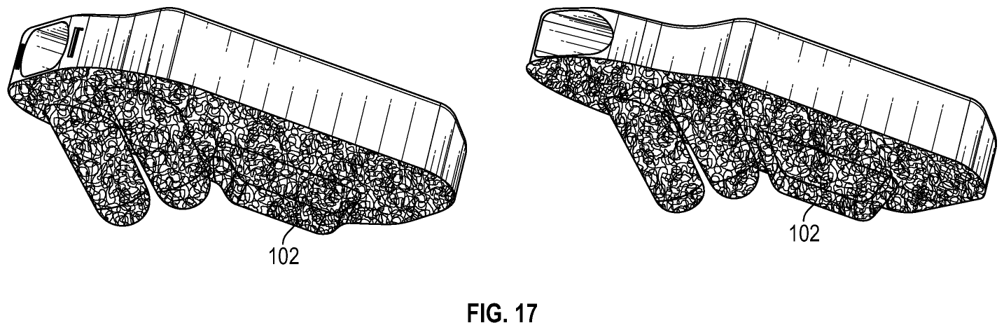

FIG. 17 illustrates bottom perspective views of a baseplate according to various embodiments. The baseplates may comprise a porous surface 102. The porous surface 102 may be configured to promote bone in growth to complement other features configured to secure the baseplate to a talus. The porous surface 102 may be configured as a trabecular structure configured to promote bone in-growth. Additionally, or alternatively, the porous surface 102 may be configured to be hydrophilic. Additionally, or alternatively, the porous surface 102 may be configured to include cells that are configured to receive biologic material prior to implantation to promote bone in-growth after implantation. Additionally, or alternatively, the porous surface 102 may be configured to include less metal mass implanted and more open areas for the bone to grow through the implant and create a more rigid fusion of the baseplate to a talus. In an embodiment, the porous surface 102 may be applied to a baseplate bone facing side and/or one or more protrusions configured to engage a bone portion. In an embodiment, the baseplate may be additively manufactured with integrated porous features configured for bone integration. Alternatively, the porous surface 102 may be additively manufactured with integrated porous features configured for bone integration and the porous surface 102 may be subsequently secured to a baseplate portion.

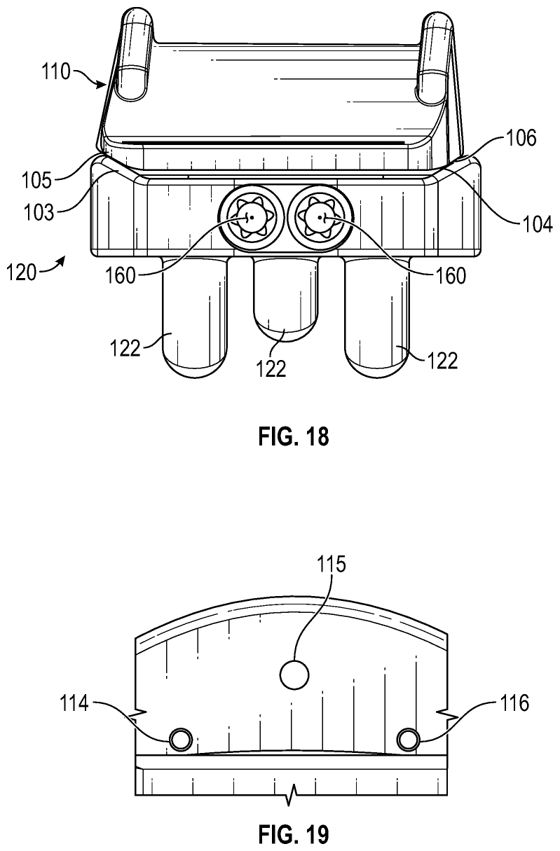

FIG. 18 illustrates a front view of an adjustable system for ankle arthroplasty 100 according to an embodiment. The adjustable system for ankle arthroplasty 100 may comprise an articulating component 110 and a baseplate 120. The articulating component 110 and baseplate 120 may be configured so that when the articulating component 110 is engaged with the baseplate 120 the articulating component bone facing side is centered on the baseplate joint facing side. The baseplate joint facing side may comprise a plurality of first angled surfaces and the articulating component bone facing side may comprise a plurality of second angled surfaces configured to slidably engage the plurality of first angled surfaces such that the articulating component is centered on the baseplate in a medial-lateral direction. The baseplate 120 may include a first baseplate centering surface 103 and a second baseplate centering surface 104. The articulating component 110 may include a first articulating component centering surface 105 and a second articulating component centering surface 106. The first baseplate centering surface 103 and second baseplate centering surface 104 may be configured to slidably engage the first articulating component centering surface 105 and the second articulating component centering surface 106. In some embodiments, actuation of a first locking feature 130 and a second locking feature 150 may result in a force applied to the articulating component in a caudal direction, which may result in engagement of the first baseplate centering surface 103 and second baseplate centering surface 104 with the first articulating component centering surface 105 and the second articulating component centering surface 106. The geometries of the first baseplate centering surface 103; second baseplate centering surface 104; first articulating component centering surface 105; and the second articulating component centering surface 106 may be configured such that the resulting caudal force centers the articulating component 110 on the baseplate 120.

FIG. 19 illustrates a partial side view of an adjustable system for ankle arthroplasty 100 according to an embodiment. The adjustable system for ankle arthroplasty 100 may comprise an articulating component 110. In an embodiment, an articulating component 110 may further comprise an apex visualization aperture 115 configured to facilitate visualization through the articulating component 110 in a medial-lateral direction. Alternately, the apex visualization aperture 115 may facilitate visualization through radiographic means. The apex visualization aperture 115 may be configured to provide a means of confirming the orientation of the articulating component 110 in both an anterior-posterior plane and a cephalad-caudal plane.

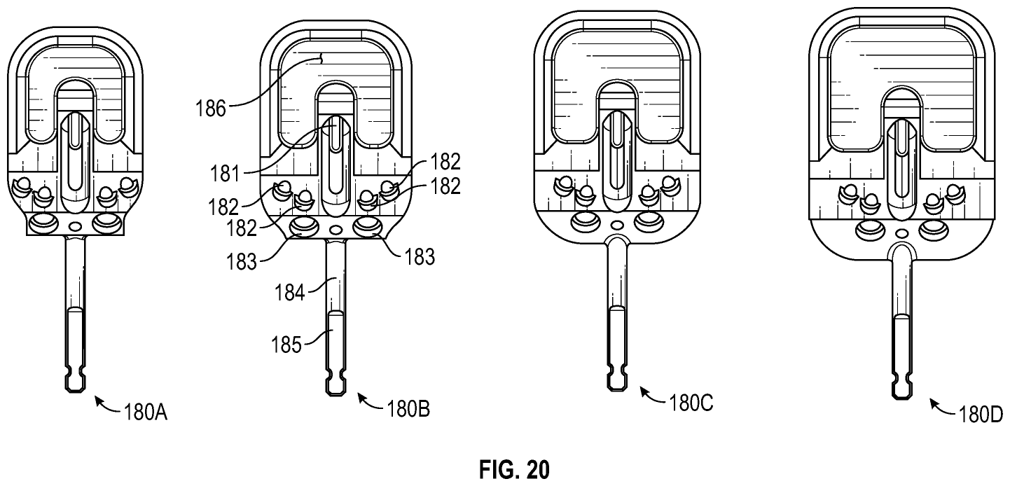

FIG. 20 illustrates top views of a trialing and peg drilling & keel reaming instrument according to various embodiments. The trialing and peg drilling & keel reaming instrument 180a, 180B, 180C, 180D may be configured to perform various functions that may be performed at various steps of a surgical procedure. The various configurations may be configured to complement a range of patient anatomies. The trialing and peg drilling & keel reaming instrument 180a, 180B, 180C, 180D may comprise one or more keel reaming guide slots 181; one or more guide pin apertures 182, one or more talar peg guide apertures 183; a handle 184; a quick connect feature 185; and a body aperture 186. The handle 184 and the quick connect feature 185 may be configured to detachably connect to a hand piece or other instrument to facilitate placement of the trialing and peg drilling & keel reaming instrument 180a, 180B, 180C, 180D and/or facilitate stabilization of the trialing and peg drilling & keel reaming instrument 180a, 180B, 180C, 180D during use. The trialing and peg drilling & keel reaming instrument 180a, 180B, 180C, 180D may be configured to be detachably secured to a talus using guide wires 190. In an embodiment, the one or more guide pin apertures 182 may be configured to receive one or more guide wires 190.

The trialing and peg drilling & keel reaming instrument 180a, 180B, 180C, 180D may be configured as a size trial to assess a patient's anatomy to determine a desired implant size and/or configuration. The body aperture 186 may be configured to facilitate visualization of a patient's anatomy when the trialing and peg drilling & keel reaming instrument 180a, 180B, 180C, 180D is within a surgical site.

Additionally, or alternatively, the trialing and peg drilling & keel reaming instrument 180a, 180B, 180C, 180D may be configured as a peg drilling guide. The trialing and peg drilling & keel reaming instrument 180a, 180B, 180C, 180D may include one or more talar peg guide apertures 183. The size, location, and angle of the one or more talar peg guide apertures 183 may be configured to align with one or more talar pegs 122 of an embodiment of a baseplate 120.

Additionally, or alternatively, the trialing and peg drilling & keel reaming instrument 180a, 180B, 180C, 180D may be configured as a keel reaming guide. The trialing and peg drilling & keel reaming instrument 180a, 180B, 180C, 180D may include one or more keel reaming guide slots 181. The size, location, and angle of the one or more keel reaming guide slots 181 may be configured to align with one or more keels 121 of an embodiment of a baseplate 120.

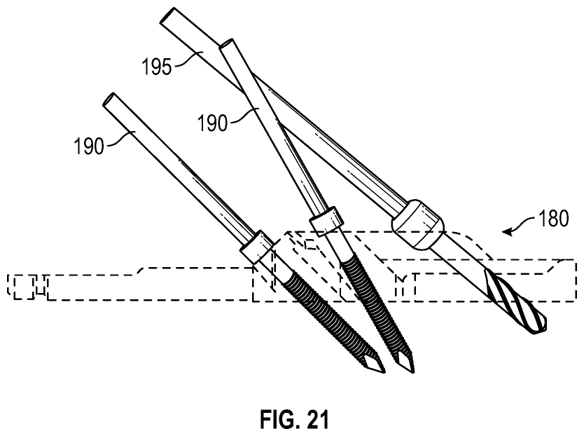

FIG. 21 illustrates a side view of a drill 195; a guide wire 190; and a trialing and peg drilling & keel reaming instrument 180 according to an embodiment. The trialing and peg drilling & keel reaming instrument 180a, 180B, 180C, 180D may comprise a plurality of apertures configured to receive a cutting instrument. In an embodiment, the guide wire 190 may include a depth stop feature to limit the cutting depth of the guide wire 190 through a trialing and peg drilling & keel reaming instrument 180a, 180B, 180C, 180D, into a talus. In an embodiment, the drill 195 may include a depth stop feature to limit the cutting depth of the drill 195 through a trialing and peg drilling & keel reaming instrument 180a, 180B, 180C, 180D, into a talus.

In an embodiment, a method for implanting an ankle arthroplasty device, such as the adjustable system for ankle arthroplasty 100, may include the following steps:
1. Perform talar bone resection;
2. Remove talar bone resection;
3. Use trialing and peg drilling & keel reaming instrument to determine optimum size and configuration of adjustable system for ankle arthroplasty;
4. Place chosen size/configuration of trialing and peg drilling & keel reaming instrument into position on talus and secure with guide wires;
5. Drill holes for talar pegs, if necessary, using drill and trialing and peg drilling & keel reaming instrument;
6. Ream slot for keel, if necessary, using drill and trialing and peg drilling & keel reaming instrument;
7. Remove guide wires and drill and trialing and peg drilling & keel reaming instrument;
8. Place articulating component onto baseplate and secure baseplate to a talus; alternately, secure baseplate to a talus and then place articulating component onto baseplate;
9. Use an apex visualization aperture and a radiographic device to confirm orientation and location of the adjustable system for ankle arthroplasty, adjust as necessary;
10. Use actuators in the baseplate to adjust the position of the articulating component in the anterior-posterior plane to achieve desired alignment and/or engagement an inferior articular surface of a tibia;
11. Use actuators in the baseplate to lock the position of the articulating component using locking features;
12. Use one or more visualization apertures and a radiographic device to confirm locking features are in a locked position;
13. Secure actuator locking mechanism within baseplate cavity.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. An adjustable system for ankle arthroplasty, the adjustable system comprising:
a baseplate comprising:
a baseplate bone facing side configured to be secured to a talus; and
a baseplate joint facing side; and
an articulating component comprising:
an articulating component joint facing side comprising a prosthetic talar articular surface; and
an articulating component bone facing side configured to adjustably engage the baseplate joint facing side;
wherein the baseplate and the articulating component cooperate to define a locking mechanism configured to be actuated to secure the articulating component bone facing side to the baseplate joint facing side.

2. The adjustable system of claim 1, wherein the baseplate bone facing side is additively manufactured with integrated porous features configured for bone integration.

3. The adjustable system of claim 1, wherein the articulating component is configured for both right ankle arthroplasty and left ankle arthroplasty.

4. The adjustable system of claim 1, wherein the locking mechanism includes a polymer anti-rotation component.

5. An adjustable system for ankle arthroplasty, the adjustable system comprising:
a baseplate comprising:
a baseplate bone facing side configured to be secured to a talus; and
a baseplate joint facing side; and
an articulating component comprising:
an articulating component joint facing side comprising a prosthetic talar articular surface; and
an articulating component bone facing side configured to be secured to the baseplate joint facing side at any of a plurality of relative positions.

6. The adjustable system of claim 5, wherein the articulating component bone facing side is configured to be secured to the baseplate joint facing side at any of a plurality of relative positions in-vivo and in-vitro.

7. The adjustable system of claim 5, wherein the articulating component bone facing side is configured to be secured to the baseplate joint facing side at any of a plurality of relative positions along an anterior-posterior direction.

8. The adjustable system of claim 5, wherein:
the baseplate joint facing side comprises a plurality of first angled surfaces; and
the articulating component bone facing side comprises a plurality of second angled surfaces configured to slidably engage the plurality of first angled surfaces such that the articulating component is centered on the baseplate in a medial-lateral direction.

9. An adjustable system for ankle arthroplasty, the adjustable system comprising:
a baseplate comprising:
a baseplate bone facing side configured to be secured to one of a talus and a tibia; and
a baseplate joint facing side; and
an articulating component configured to be secured to the baseplate such that the articulating component can be moved along the baseplate without detaching the articulating component from the baseplate, the articulating component comprising:
an articulating component joint facing side comprising a prosthetic articular surface; and
an articulating component bone facing side.

10. The adjustable system of claim 9, wherein the baseplate bone facing side further comprises one or more protrusions configured to engage a bone.

11. The adjustable system of claim 9, wherein:
the baseplate and the articulating component cooperate to define a locking mechanism configured to secure the articulating component to the baseplate, the locking mechanism comprising:
a plurality of baseplate locking features of the baseplate; and
a plurality of articulating component locking features of the articulating component; and
the plurality of articulating component locking features is configured to slidably mate with the plurality of baseplate locking features at any of a plurality of relative positions of the baseplate and the articulating component along an anterior-posterior direction.

12. The adjustable system of claim 11, wherein the plurality of baseplate locking features comprises:
a first baseplate dovetail feature that is adjustably positionable along the anterior-posterior direction; and
a second baseplate dovetail feature that is adjustably positionable, independently of a position of the first baseplate dovetail feature, along the anterior-posterior direction.

13. The adjustable system of claim 12, wherein the plurality of baseplate locking features further comprises:
a first actuator configured to slidably adjust the first baseplate dovetail feature; and
a second actuator configured to slidably adjust the second baseplate dovetail feature.

14. The adjustable system of claim 13, wherein the first actuator and the second actuator are configured to be captive within the baseplate.

15. The adjustable system of claim 14, wherein the first actuator and the second actuator comprise a threaded portion.

16. The adjustable system of claim 13, wherein the plurality of baseplate locking features further comprises an actuator locking mechanism configured to lock a position of the first actuator and the second actuator.

17. The adjustable system of claim 16, wherein the actuator locking mechanism is configured to captively snap into the baseplate.

18. The adjustable system of claim 11, wherein the baseplate comprises one or more apertures configured to facilitate visualization of the plurality of baseplate locking features when the plurality of baseplate locking features are in a locked position.

19. The adjustable system of claim 9, wherein the articulating component comprises one or more apertures configured to facilitate visualization through the articulating component in a medial-lateral direction.

20. The adjustable system of claim 9, further comprising a size trial comprising a plurality of apertures configured to receive a cutting instrument.
